(19) United States
(12) Patent Application Publication
(10) Pub. No.: US 2008/0208298 A1
(43) Pub. Date: Aug. 28, 2008

(54) THERAPEUTIC UPPER BODY WRAP

(76) Inventor: Hagay Mizrahi, Woodland Hills, CA (US)

Correspondence Address: BELASCO, JACOBS & TOWNSLEY LLP;HOWARD HUGHES CENTER, 6100 CENTER DRIVE, SUITE 630, LOS ANGELES, CA, 90045, US

(21) Appl. No.: 11/680,528

(22) Filed: Feb. 28, 2007

(51) Int. Cl. A61F 7/00

(52) U.S. Cl. 607/108; 607/112; 607/114

(57) ABSTRACT

A therapeutic upper body wrap has a flexible, aroma-permeable cover having upper and lower layers joined at their outer edges to form an interior space to contain particulate matter which may include thermal reservoir and aromatherapy materials. The interior space is divided into a series of subspaces in the form of parallel, tube-shaped containers to prevent shifting of the materials. A central opening is provided penetrating the upper and layers of the cover. The opening permits a user to place the wrap over his head and onto his shoulders. The openings are substantially circular, designed to fit snugly about a user's neck. A slit is provided extending from the outer edge of the wrap to the opening for easy placement about the neck. A fastener, such as a hook and loop, button, zipper or tie string is used to close the slit. The wrap is heated or cooled before use.

### FIELD OF THE INVENTION

The invention pertains to devices for alleviating muscle soreness, fatigue and stress. More particularly, the invention relates to herb packs that can be heated or cooled and then applied to the body to provide relief through temperature difference and aromatherapy related to the herbs contained in a wearable pad.

### BACKGROUND OF THE INVENTION

Heating and cooling pads have long been in use for alleviating soreness, stiffness, aching muscles and the results of various types of injuries. Likewise, aromatherapy products have been in use in Eastern cultures for hundreds of years and are now achieving prominence in the West. The combination of heating and cooling devices with aromatherapy products has served to provide increased benefits for those with muscle, tendon and joint problems. A number of inventions have been developed that use one or more of these components to alleviate pain or soreness.

U.S. Pat. No. 6,699,271, issued to Clayton, describes a therapeutic wrap designed to be applied to various portions of an individual, such as the neck and shoulders. The wrap contains a plurality of narrow channels into which a filler material, such as Basmati rice, is provided. Various herbs such as chamomile or lavender are also provided within each of the channels to allow for aromatherapy. The rice serves as heat-transferring material. Each channel is separated from its adjoining channel by stitching it closed such. The neck wrap is designed to encircle the neck of the user and fasten with Velcro fasteners. The Velcro fasteners and the use of narrow channels keep the filler material within the wrap from shifting and hold it close to the neck during treatment. Basmati rice and aromatic herbs are the preferred fillers. In use, the wrap is heated by placing it in a conventional oven, crockpot or microwave, or cooled by placing it into a freezer or refrigerator.

U.S. Pat. No. 5,476,492, issued to Unrug, discloses a body warmer for therapeutic purposes containing whole herb seed. The body warmer is comprised of a shell of an elastic breathable and pliable material, divided into chambers containing rebufacient herbal material in the form of whole seed grains, and means of attachment to keep the warmer in place in a desired area of the human body. The shell is made in two layers of fabric fastened along two long sides at side seams. Other seams divide the shell into a number of tubular chambers filled with herb seed. Ribbon tie straps attached to corners of the shell are used to fasten the shoulder warmer pad to the body.

U.S. Pat. No. 5,603,727, issued to Clark et al., is directed to a thermal pack having temperature-retaining particles within the packet. A plurality of barriers located within the packet form migration paths and retaining areas. The pack that has pliable container consisting of top and bottom surfaces connected about their periphery. Barriers within the compartment to direct the migration of the heat retaining particles in the compartment are formed by sewing portions of the top and bottom surfaces together in a chevron shape. Heat retaining particles are preferably re-cleaned animal feed corn, which can be scented. The thermal material can be either heated or cooled depending on the appropriate treatment.

U.S. Pat. No. 5,948,010, issued to Adamee, discloses a therapeutic heat application device. The device comprises a pad portion having a wide central portion divided into a plurality of narrow sections by vertically extending seams. Each of the tapered side portions are divided into two sections by a horizontally extending seam. The sections of the central portion and side portions are filled with a granular natural filler material where the seams would maintain the proper positioning of the filler material throughout the pad portion. A pair of straps have hook and loop fasteners for fastening the pad around the body of the wearer. In use, the device would be heated in a microwave oven or chilled for cold therapy.

U.S. Pat. No. 5,890,487, issued to Kimmel, is directed to a flat fabric bag that holds a quantity of dried Indian corn, which can be scented. The bag can then be heated in a microwave oven or cooled in a freezer. The heating pad that is formed by sewing two pieces of cloth together to form closed bag which is then filled with corn. To keep the corn spread throughout the bag, a number of baffle stitches are used which divide the bag into sections that hold a uniform quantity of corn. The bag is used primarily over the shoulders, where the wide end portions drape over the tops of the shoulders and the long center portion runs along the upper back or neck.

It is an objective of the present invention to provide a means to provide a means to alleviate muscle soreness, cramps, aches and pains, etc. without the use of drugs or invasive procedures. It is a further objective to provide a pad that provides these benefits that can be comfortably and easily worn by a user. It is a still further objective of the invention to provide a pad that can provide both heating and cooling to the body. It is yet a further objective to provide a pad that includes the benefits of aromatherapy for the user. Finally, it is an objective of the invention to provide a pad with the above benefits that is easily attached and removed.

While some of the objectives of the present invention are disclosed in the prior art, none of the inventions found include all of the requirements identified.

### SUMMARY OF THE INVENTION

The present invention addresses all of the deficiencies of heating and cooling pack inventions and satisfies all of the objectives described above.

(1) A therapeutic upper body wrap providing the desired features may be constructed from the following components. A flexible, aroma-permeable cover is provided. The cover is adapted for placement about a neck and on to shoulders, chest and back of a user. The cover has an upper layer and a lower layer. The upper and lower layers are joined at their outer edges and define a space between them. The space is divided into a plurality of subspaces.

An opening is provided. The opening is centrally located in the cover, sized to fit closely about the neck of the user, penetrating the upper and lower layers, and forming endpoints for at least some of the subspaces. An access slit is provided. The slit extends from the opening to an outer edge of the cover. A quantity of particulate material is provided. The particulate material is secured within the subspaces.

(2) In a variant of the invention, the space is divided into a plurality of subspaces with stitching.

(3) In another variant, at least some of the particulate material is thermal reservoir material.

(4) In still another variant, at least some of the particulate material is aromatherapy material.

(5) In yet another variant, the subspaces are a series of tube-shaped containers.

(6) In a further variant, the tube-shaped containers are divided into substantially equal portions to prevent shifting of the particulate material.

(7) In still a further variant, a fastener is provided. The fastener closes the slit to secure the wrap about the upper body of the user.

(8) In yet a further variant, the fastener is selected from the group consisting of button, hook and loop fastener, zipper and tie string.

(9) In another variant of the invention, the thermal reservoir materials are selected from the group consisting of corn, rice, wheat, oats, barley, beans and flaxseed.

(10) In yet another variant, the aromatherapy materials are selected from the group consisting of cinnamon, chamomile, lemongrass, peppermint, rosemary, spearmint, valerian root, white willow, yarrow, lavender ginger and yellow dock root.

(11) In still another variant, the subspaces have openable closures to permit introduction of alternative particulate materials.

(12) In a final variant, the openable closures are selected from the group consisting of hook and loop fastener, zipper, button and tie string.

An appreciation of the other aims and objectives of the present invention and an understanding of it may be achieved by referring to the accompanying drawings and the detailed description of a preferred embodiment.

### DESCRIPTION OF THE DRAWINGS

FIG. 1 is a perspective view of the preferred embodiment of the invention disposed about the neck and shoulders of a user;

FIG. 2 is a perspective view of the of the FIG. 1 embodiment illustrating the head opening and access slit;

FIG. 3 is a partial cutaway perspective illustrating the tube-shaped subspaces containing thermal reservoir materials and aromatherapy materials; and

FIG. 4 is a perspective view of the of an alternative embodiment having a close-fitting neck opening and a reclosable slit extending from one edge of the opening to an outer edge of the wrap across the tube-shaped subspaces.

### DETAILED DESCRIPTION OF THE PREFERRED EMBODIMENT

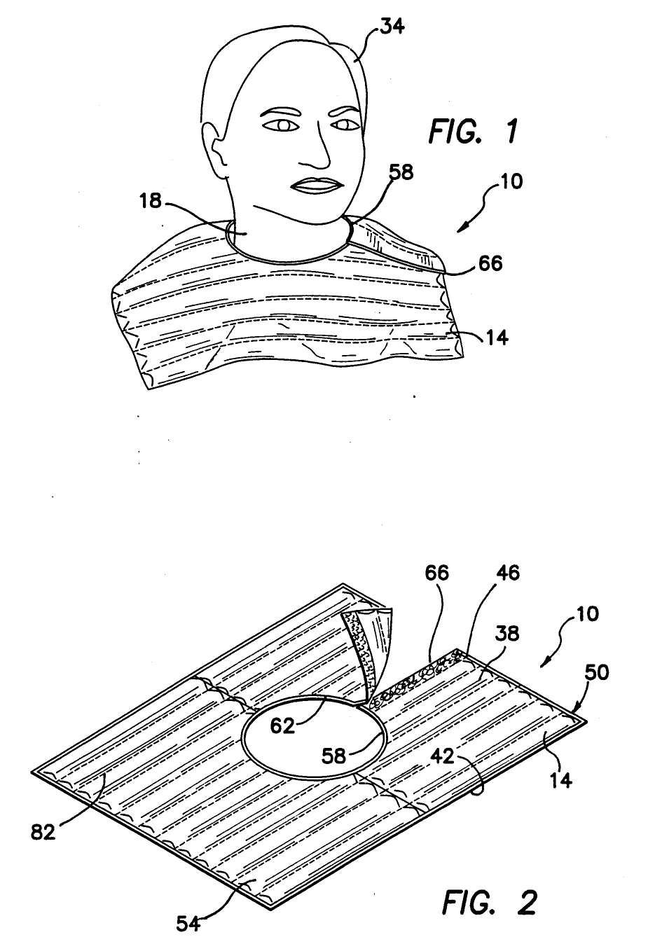

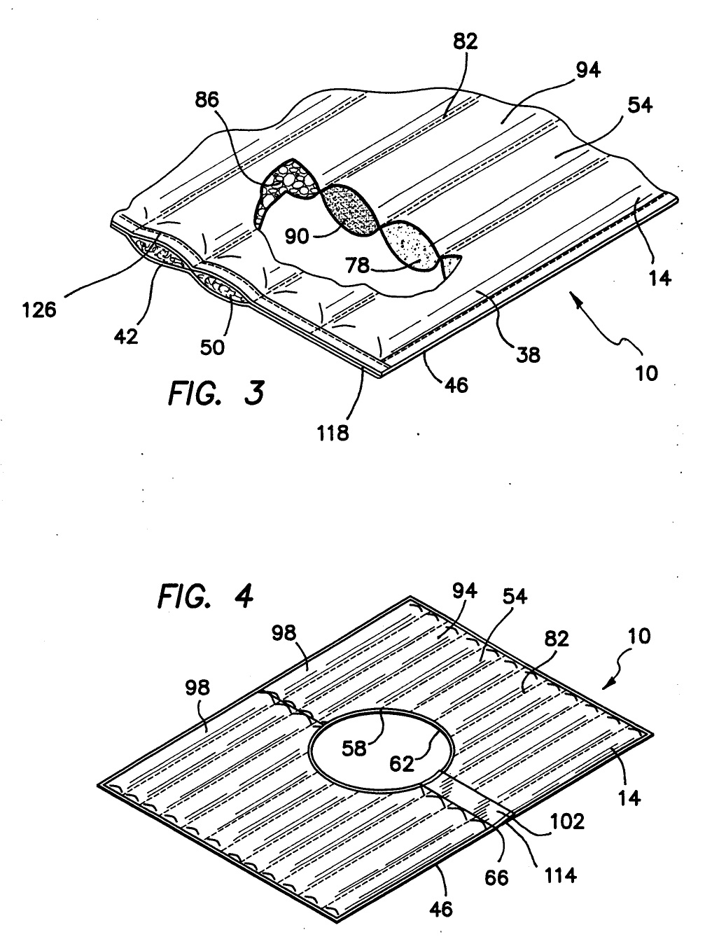

(1) FIGS. 1-4 illustrate a therapeutic upper body wrap 10 providing the desired features that may be constructed from the following components. A flexible, aroma-permeable cover 14 is provided. The cover 14 is adapted for placement about a neck 18 and on to shoulders (not shown), chest (not shown) and back (not shown) of a user 34. The cover 14 has an upper layer 38 and a lower layer 42. The upper 38 and lower 42 layers are joined at their outer edges 46 and define a space 50 between them. The space 50 is divided into a plurality of subspaces 54.

An opening 58 is provided. The opening 58 is centrally located in the cover 14, sized to fit closely about the neck 18 of the user 34, penetrating the upper 38 and lower 42 layers, and forming endpoints 62 for at least some of the subspaces 54. An access slit 66 is provided. The slit 66 extends from the opening 58 to the outer edge 46 of the cover 14. A quantity of particulate material 78 is provided. The particulate material 78 is secured within the subspaces 54.

(2) In a variant of the invention, the space 50 is divided into a plurality of subspaces 54 with stitching 82.

(3) In another variant, at least some of the particulate material 78 is thermal reservoir material 86.

(4) In still another variant, at least some of the particulate material 78 is aromatherapy material 90.

(5) In yet another variant, the subspaces 54 are a series of tube-shaped containers 94.

(6) In a further variant, the tube-shaped containers 94 are divided into substantially equal portions 98 to prevent shifting of the particulate material 78.

(7) In still a further variant, a fastener 102 is provided. The fastener 102 closes the slit 66 to secure the wrap 10 about the neck 18 of the user 34.

(8) In yet a further variant, the fastener 102 is selected from the group consisting of button (not shown), hook and loop fastener 114, zipper (not shown) and tie string (not shown).

(9) In another variant of the invention, the thermal reservoir materials 86 are selected from the group consisting of corn, rice, wheat, oats, barley, beans and flaxseed.

(10) In yet another variant, the aromatherapy materials 90 are selected from the group consisting of cinnamon, chamomile, lemongrass, peppermint, rosemary, spearmint, valerian root, white willow, yarrow, lavender ginger and yellow dock root.

(11) In still another variant, the subspaces 54 have openable closures 126 to permit introduction of alternative particulate materials 78.

(12) In a final variant, the openable closures 126 are selected from the group consisting of hook and loop fastener (not shown), zipper 118, button (not shown) and tie string (not shown).

The therapeutic upper body wrap 10 has been described with reference to particular embodiments. Other modifications and enhancements can be made without departing from the spirit and scope of the claims that follow.

1. A therapeutic upper body wrap, comprising:
a flexible, aroma-permeable cover, said cover being adapted for placement about a neck and on to shoulders, chest and back of a user, having an upper layer and a lower layer, said upper and lower layers being joined at outer edges thereof and defining a space therebetween, said space being divided into a plurality of subspaces;
an opening, said opening being centrally located in said cover, sized to fit closely about said neck of said user, penetrating said upper and lower layers, and forming endpoints for at least some of said subspaces;
an access slit, said slit extending from said opening to an outer edge of said cover;
a quantity of particulate material; and
said particulate material being secured within said subspaces.

2. The therapeutic upper body wrap, as described in claim 1, wherein said space is divided into a plurality of subspaces with stitching.

3. The therapeutic upper body wrap, as described in claim 1, wherein at least some of said particulate material is thermal reservoir material.

4. The therapeutic upper body wrap, as described in claim 1, wherein at least some of said particulate material is aromatherapy material.

5. The therapeutic upper body wrap, as described in claim 1, wherein said subspaces are a series of tube-shaped containers.

6. The therapeutic upper body wrap, as described in claim 5, wherein said tube-shaped containers are divided into substantially equal portions to prevent shifting of said particulate material.

7. The therapeutic upper body wrap, as described in claim 1, further comprising a fastener, said fastener closing said slit to secure said wrap about the upper body of said user.

8. The therapeutic upper body wrap, as described in claim 7, wherein said fastener is selected from the group consisting of:
button, hook and loop fastener, zipper and tie string.

9. The therapeutic upper body wrap, as described in claim 3, wherein said thermal reservoir materials are selected from the group consisting of:
corn, rice, wheat, oats, barley, beans and flaxseed.

10. The therapeutic upper body wrap, as described in claim 4, wherein said aromatherapy materials are selected from the group consisting of:
cinnamon, chamomile, lemongrass, peppermint, rosemary, spearmint, valerian root, white willow, yarrow, lavender ginger and yellow dock root.

11. The therapeutic upper body wrap, as described in claim 1, wherein said subspaces have openable closures to permit introduction of alternative particulate materials.

12. The therapeutic upper body wrap, as described in claim 11, wherein said openable closures are selected from the group consisting of:
hook and loop fastener, zipper, button and tie string.
